(19) United States
(12) Patent Application Publication
(10) Pub. No.: US 2006/0182773 A1
(43) Pub. Date: Aug. 17, 2006

(54) Emulsifier composition and transparent or translucent micro emulsions containing said emulsifier composition

(76) Inventors: Stefan Bruning, Philadelphia, PA (US); Achlm Ansmann, Erkrath (DE); Kathrin Tappe, Monchengladbach (DE); Bettina Jackwerth, Langerfeld (DE)

Correspondence Address: COGNIS CORPORATION;PATENT DEPARTMENT, 300 BROOKSIDE AVENUE, AMBLER, PA, 19002, US

(21) Appl. No.: 10/546,282

(22) PCT Filed: Dec. 19, 2003
(86) PCT No.: PCT/EP03/14601

§ 371 Date: Aug. 19, 2005

(30) Foreign Application Priority Data
Feb. 20, 2003 (DE) 102 07 10.4

(51) Int. Cl. A61K 8/06; B01F 17/14

(52) U.S. Cl. 424/401; 516/74

(57) ABSTRACT

The disclosed invention relates to an emulsifier composition containing hydrophilic and lipophilic emulsifiers, characterized in that the hydrophilic emulsifier is selected from at least one of the compounds of group (A) which consists of (A.1) products of the addition of 8 to 30 moles ethylene oxide onto C₁₆₋₂₂ fatty alcohols, (A.2) products of the addition of 5 to 60 moles ethylene oxide onto C₁₂₋₁₈ fatty acid triglycerides, (A.3) products of the addition of 10 to 30 moles ethylene oxide onto sorbitan mono-C₁₂₋₁₈-fatty acid esters, (A.4) products of the addition of 10 to 30 moles ethylene oxide onto C₈₋₁₈ fatty acid partial glycerides, and (A.5) products of the addition of 1 to 10 moles ethylene oxide and 1 to 5 moles propylene oxide onto C₁₀₋₁₈ fatty alcohols; and the lipophilic emulsifier is selected from at least one of the compounds of group (B) which consists of (B.1) mono- and diesters of C₁₆₋₁₈ fatty acids with glycerol and (B.2) C₁₆₋₂₂ fatty alcohols, with the proviso that, based on the total quantity of hydrophilic emulsifier, at least 38% by weight of the compounds selected from group (A) are emulsifiers formed by addition of at least 20 moles ethylene oxide and that the hydrophilic emulsifier is present in a quantity of 65 to 80% by weight, based on the total quantity of hydrophilic and lipophilic emulsifiers. The disclosed invention also includes a microemulsion containing the emulsifying agent. The emulsifying agent and emulsifiers containing it can be used, particularly, in cosmetic preparations and in the production of deodorizing compositions.

### FIELD OF THE INVENTION

This invention relates to emulsifier compositions and to transparent or translucent microemulsions containing them. The emulsifier compositions and the emulsions containing them are particularly suitable for use in the field of cosmetic compositions and, above all, for the production of deodorizing compositions.

### PRIOR ART

Microemulsions are optically isotropic, thermodynamically stable systems which contain a water-insoluble oil component, emulsifiers and water. The clear or transparent appearance of microemulsions is a result of the small particle size of the dispersed emulsion droplets which, for the most part, is under 300 nm, fine-droplet microemulsions brown-red in transmitted light and a shimmering blue in reflected light occurring in the 100 to 300 nm range and optically clear microemulsions occurring in the sub-100 nm range. The droplet size of the macroemulsions is for the most part above 300 nm. By virtue of their greater stability in relation to macroemulsions, the finer distribution of the inner phase, their generally greater effectiveness and the better transdermal penetration of the active principles incorporated therein, microemulsions have acquired considerable significance in the formulation of cosmetic and pharmaceutical preparations. In addition, transparent or translucent microemulsions are also desirable for their optically advantageous properties.

WO-A-96/28131 describes oil-in-water emulsions which are said to have a low emulsifier content to prevent the emulsions from conveying a feeling of tackiness. In addition, the microemulsions are said to be transparent or translucent. In fact, however, the emulsifiers described in WO-A-96/28131 comprise virtually all the o/w emulsifiers typical of the prior art which, moreover, can be combined with one another as required. At best a small fraction of the immense number of selection possibilities covered by WO '131 actually leads to microemulsions which possess the alleged properties. However, WO '131 does not show in what way a suitable choice could be made for the production of transparent, non-tacky microemulsions. On the whole, therefore, WO '131 does not impart a clear and reproducible teaching for the production of microemulsions having the claimed properties.

There is thus still a need for transparent or translucent microemulsions which, in addition, create a pleasant sensory impression on the skin with little feeling of tackiness and for microemulsions whose viscosity is adjustable over a broad range.

Accordingly, the problem addressed by the present invention was to provide microemulsions having the properties described above and emulsifier compositions for the production of such microemulsions.

### DESCRIPTION OF THE INVENTION

The problem stated above has been solved by the emulsifier composition claimed in claim 1 and by the microemulsion claimed in claim 8. The invention also relates to the use of the emulsifier composition claimed in claim 7 and to the use of the microemulsion claimed in claim 15. Preferred embodiments and further developments of the invention are described in the subsidiary claims.

Accordingly, in a first embodiment, the present invention relates to an emulsifier composition containing hydrophilic and lipophilic emulsifiers, characterized in that the hydrophilic emulsifier is selected from at least one of the compounds of group (A) which consists of
(A.1) products of the addition of 8 to 30 mol ethylene oxide onto C₁₆₋₂₂ fatty alcohols,
(A.2) products of the addition of 5 to 60 mol ethylene oxide onto C₁₂₋₁₈ fatty acid triglycerides,
(A.3) products of the addition of 10 to 30 mol ethylene oxide onto sorbitan mono-C₁₂₋₁₈-fatty acid esters,
(A.4) products of the addition of 10 to 30 mol ethylene oxide onto C₈₋₁₈ fatty acid partial glycerides and
(A.5) products of the addition of 1 to 10 mol ethylene oxide and 1 to 5 mol propylene oxide onto C₁₀₋₁₈ fatty alcohols,
   and the lipophilic emulsifier is selected from at least one of the compounds of group (B) which consists of
(B.1) mono- and diesters of C₁₋₁₈ fatty acids with glycerol and
(B.2) C₁₂₂ fatty alcohols,
   with the proviso that, based on the total quantity of hydrophilic emulsifier, at least 38% by weight of the compounds selected from group (A) are emulsifiers formed by addition of at least 20 mol ethylene oxide and that the hydrophilic emulsifier is present in a quantity of 65 to 80% by weight, based on the total quantity of hydrophilic and lipophilic emulsifiers.

The special combination of lipophilic and hydrophilic emulsifiers in the quantity ranges mentioned ensures that the emulsifier composition according to the invention leads to transparent or translucent oil-in-water microemulsions with virtually any of the oil phases typically encountered in pharmaceutical and cosmetic compositions and particularly in deodorizing compositions. Particular significance attaches to the high percentage of hydrophilic emulsifiers in the emulsifier composition. On the one hand, the percentage of hydrophilic emulsifiers of group (A) amounts to at least 65% by weight of the emulsifiers as a whole and, on the other hand, a high percentage of highly ethoxylated emulsifiers is present in group (A). The increased hydrophilic character of the composition according to the invention in relation to conventional emulsifier compositions is regarded as the reason why stable, translucent or transparent microemulsions with very little, if any, tackiness are formed.

Hydrophilic Emulsifier

In a particularly preferred embodiment, the hydrophilic emulsifier is selected from at least one of the compounds of group (A) which consists of
(A.1) products of the addition of 10 to 25 mol ethylene oxide onto C₁₆₋₂₂ fatty alcohols,
(A.2) reaction products of 30 to 60 mol ethylene oxide with castor oil, hydrogenated castor oil or almond oil,
(A.3) products of the addition of 15 to 25 mol ethylene oxide onto sorbitan mono-C₁₆₋₁₈-fatty acid esters, more particularly sorbitan mono-C₁₈-fatty acid esters,
(A.4) products of the addition of 15 to 25 mol ethylene oxide onto C₈₋₁₂ fatty acid partial glycerides, more particularly C₁₂ fatty acid partial glycerides, and
(A.5) products of the addition of 1 to 10 mol ethylene oxide and 1 to 5 mol propylene oxide onto C₁₀₋₁₄ fatty alcohols, more particularly C₁₂ fatty alcohols.

Products of the addition of ethylene oxide and/or propylene oxide onto fatty alcohols, sorbitan esters, fatty acid partial esters or onto triglycerides, such as castor oil, are known, commercially available products. They are homolog mixtures of which the average degree of alkoxylation corresponds to the ratio between the quantities of ethylene oxide and/or propylene oxide and substrate with which the addition reaction is carried out.

Fatty alcohols which give the compounds (A.1) by addition of ethylene oxide are understood in particular to be primary aliphatic alcohols, such as cetyl alcohol, palmitoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and the technical mixtures thereof formed, for example, in the high-pressure hydrogenation of technical methyl esters based on fats and oils or aldehydes from Roelen's oxo synthesis and as monomer fraction in the dimerization of unsaturated fatty alcohols. Suitable emulsifiers from the group of ethoxylated fatty alcohols are commercially obtainable, for example, as Eumulgin® B1 (Ceteareth-12); Eumulgin® B2 (Ceteareth-20), Eumulgin® B3 (Ceteareth-30), Eumulgin® BA 10 (Beheneth-10), Eumulgin® BA 25 (Beheneth-25), Eumulgin® O 10 (Oleth-10), Eumulgin® O 20 (Cetoleth-20), Eumulgin® O 30 (Oleth-30), Eumulgin® RO 40 (PEG-40 Castor Oil) from Cognis Deutschland GmbH & Co. KG.

Suitable ethoxylated partial glycerides are the ethoxylation products of mono- or diglycerides of C₈₋₁₈ fatty acids with branched or linear alkyl groups, preferably linear alkyl groups. Examples of suitable fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, ricinoleic acid and technical mixtures thereof. The partial glycerides may still contain small quantities of triglyceride from their production. Ethoxylated partial glycerides of C₈₋₁₂ fatty acids are preferred, those of lauric acid being particularly preferred. A PEG-20 Glyceryl Laurate is obtainable, for example, under the name of Lamacit® GML 20 from Cognis Deutschland GmbH & Co. KG.

The residues of the C₁₂₋₁₈ acids mentioned in connection with the ethoxylated partial glycerides may be used as the fatty acid components of the ethoxylated sorbitan esters. Sorbitan monooleate is particularly preferred. Examples of commercially obtainable ethoxylated sorbitan esters are Eumulgin® SML 20 (Polysorbate 20) and Eumulgin® SMO 20 (Polysorbate 80) of Cognis Deutschland GmbH & Co. KG.

The fatty acid components of the ethoxylated triglycerides may also be derived from the C₁₂₋₁₈ fatty acids mentioned in connection with the ethoxylated partial glycerides. However, the compounds of group (A.2) are preferably selected from reaction products of on average 5 to 60 mol and preferably 30 to 60 mol ethylene oxide with almond oil and, more particularly, with castor oil or hydrogenated castor oil. Cleavage products with terminal ethylene oxide groups are formed during the reaction of the oils mentioned with ethylene oxide. Corresponding emulsifiers are known in principle and are obtainable, for example, from Cognis Deutschland GmbH & Co. KG under the names of Eumulgin® HRE 40 and Eumulgin® HRE 60.

Suitable fatty alcohols for the ethylene oxide/propylene oxide addition products of group (A.5) are, above all, primary aliphatic alcohols. Besides the C₁₆₋₁₈ fatty alcohols already listed in connection with the ethylene oxide addition products of group (A.1), these are, for example, the relatively short-chain caproic alcohol, caprylic alcohol, 2-ethylhexyl alcohol, capric alcohol, lauryl alcohol, isotridecyl alcohol and myristyl alcohol. Mixed ethoxylated/propoxylated lauryl alcohol is particularly suitable. Commercially obtainable products of this group are, for example, Eumulgin® L (PPG-1-PEG-9 Lauryl Glycol Ether) and Aethoxal® B (PPG-5-Laureth-5) from Cognis Deutschland GmbH & Co. KG.

It is of particular advantage to the emulsifier composition according to the invention if the hydrophilic emulsifier is selected from at least one of the compounds of the group consisting of products of the addition of 10 to 30 mol and more particularly 12 to 25 mol ethylene oxide onto cetyl stearyl alcohol or behenyl alcohol, products of the reaction of 40 to 60 mol ethylene oxide with castor oil or hydrogenated castor oil and products of the addition of 5 to 10 mol ethylene oxide and 1 to 5 mol propylene oxide onto lauryl alcohol.

Lipophilic Emulsifier

So far as the lipophilic emulsifiers of the emulsifier composition according to the invention are concerned, reference may be made concerning the fatty acid glycerides of group (B.1) to the C₁₆₋₁₈ starting materials already mentioned in connection with the corresponding ethoxylated compounds. Suitable fatty acid glycerides are inter alia Cutina® GMS (Glyceryl Stearate) or Cutina® MD (Glyceryl Stearate) from Cognis Deutschland GmbH & Co. KG.

Suitable fatty alcohols of group (B.2) are the compounds mentioned as starting materials for the ethoxylated fatty alcohols of group (A.1). Lanette® 18 (Stearyl Alcohol), Lanette® O (Cetearyl Alcohol) or Lanette® 22 (Behenyl Alcohol) from Cognis Deutschland GmbH & Co. KG may be mentioned by way of example.

The lipophilic emulsifier is preferably selected from at least one of the compounds of the group consisting of partial glycerides of stearic acid and stearyl alcohol, cetearyl alcohol and behenyl alcohol.

Emulsifier compositions suitable above all are those which have a high percentage of highly ethoxylated emulsifiers and, based on the total quantity of hydrophilic emulsifier, 47 to 96% by weight and more particularly 52 to 96% by weight of the compounds selected from group (A) are preferably hydrophilic emulsifiers formed by addition of at least 20 mol ethylene oxide.

In another advantageous embodiment, the hydrophilic emulsifier is present in a quantity of 70 to 80% by weight and more particularly in a quantity of 74 to 78% by weight, based on the total quantity of hydrophilic and lipophilic emulsifiers.

The emulsifier composition described in the foregoing is eminently suitable for the production of transparent or translucent microemulsions. The present invention also relates to these transparent or translucent microemulsions which contain the emulsifier composition according to the invention. Microemulsions in the context of the invention are emulsions with a mean droplet size of 1 to 150 nm. Microemulsions with a mean droplet size of 1 to 100 nm and more particularly below 60 nm are preferred for the purposes of the invention. By “transparent or translucent” are meant microemulsions according to the invention with a transmission of 97 to 100%, preferably 98 to 100% and more particularly 99 to 100% (wave length: 700 nm; 1 cm layer thickness of the cell; 23° C.; Spectronic 20, manufacturer: Milton Roy Company). Preferred embodiments of the invention are clear/transparent microemulsions with a transmission of 99 to 100%.

The emulsifier composition is present in the microemulsion in a quantity of preferably 5 to 30% by weight and more particularly 10 to 25% by weight, based on the total quantity of microemulsion.

The small quantity of emulsifier required for emulsification is one of the reasons why the microemulsions according to the invention convey little, if any, feeling of tackiness and richness. The suitable quantity of emulsifier composition as usual is determined on the one hand by the particular components of the emulsifier composition and, on the other hand, by the other constituents of the emulsion. In addition, the quantity of emulsifier composition used and the quantity adaptation of the emulsifier composition and the other constituents influence the consistency of the microemulsion obtained. Sprayable to highly viscous and creamy emulsions can be obtained as required. The viscosity of the emulsion tends to increase with increasing quantity of oil phase.

Oil Components

Basically, the microemulsion according to the invention may contain any of the other constituents typically encountered in emulsions for cosmetic or pharmaceutical use. Oil components, fats and/or waxes are present in a quantity of 30 to 60% by weight, preferably 32 to 58% by weight and more particularly 49 to 58% by weight, based on the total quantity of oil components, fats and waxes and emulsifier composition.

Stable, transparent or translucent microemulsions with the above-mentioned consistencies are obtained above all when they contain the oil phase and the aqueous phase in a quantity ratio of 10 to 55% by weight: 90 to 45% by weight, preferably 15 to 50% by weight: 85 to 50% by weight and more particularly 15 to 45% by weight: 55 to 85% by weight. The quantities by weight mentioned include all oil-soluble components of the emulsion for the oil phase and all water-soluble components for the aqueous phase. The quantities of oil and water phase do of course add up to 100% by weight.

As mentioned, the components of the emulsions according to the invention which can be emulsified with the emulsifier composition according to the invention are not particularly limited. Basically, any oil components typically encountered in emulsions may be used. In a preferred embodiment, the microemulsion according to the invention contains as oil component at least one compound selected from the group consisting of esters of a C₁₆₋₂₂ fatty acid with a C₈₋₁₈ alcohol, more particularly a linear fatty acid with a linear alcohol, dialkyl carbonates with C₆₋₂₂ alkyl groups, more particularly with linear C₆₋₁₂ alkyl groups, silicone oils, aliphatic or naphthenic hydrocarbons, more particularly dialkyl cyclohexanes, and symmetrical or nonsymmetrical dialkyl ethers with C₆₋₂₂ alkyl groups, more particularly with linear C₆₋₁₂ alkyl groups.

Suitable oil components are, for example, esters of linear C₁₆₋₂₂ fatty acids with linear C₈₋₁₈ fatty alcohols, for example myristyl palmitate, myristyl stearate, myristyl isostearate, myristyl oleate, myristyl behenate, myristyl erucate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl palmitate, isostearyl stearate, isostearyl isostearate, isostearyl oleate, isostearyl behenate, isostearyl oleate, oleyl palmitate, oleyl stearate, oleyl isostearate, oleyl oleate, oleyl behenate and oleyl erucate. Also suitable are esters of linear C₁₆₋₂₂ fatty acids with branched alcohols, more particularly 2-ethyl hexanol. A preferred representative of this group is cetyl palmitate (for example Cutina CP from Cognis Deutschland GmbH & Co. KG).

Among the dialkyl carbonates, dicaprylyl carbonate—commercially available as Cetiol® CC from Cognis Deutschland GmbH & Co. KG—is particularly preferred.

Cyclomethicone and silicon methicone types may be regarded as examples of silicone oils.

Aliphatic or naphthenic hydrocarbons are, for example, squalane, squalene or—preferably—dialkyl cyclohexanes, such as diethyl hexyl cyclohexane (Cetiol® S, Cognis Deutschland GmbH & Co. KG).

Dicaprylyl ether (Cetiol® OE, Cognis Deutschland GmbH & Co. KG) is mentioned as an example of symmetrical or nonsymmetrical dialkyl ethers containing 6 to 22 carbon atoms per alkyl group, more particularly with linear C₆₋₁₂ alkyl groups.

Fats, Waxes, Other Additives

Waxes and fats which may also be used as constituents of the oil phase in the microemulsion according to the invention correspond to the compounds typically used in the prior art. Examples of such compounds can be found in applicants' DE 100 25 671 A1. The additives and auxiliaries mentioned in that document, such as consistency factors, thickeners, superfatting agents, stabilizers, polymers, UV filters, antioxidants, film formers, swelling agents, hydrotropes, preservatives, perfume oils, dyes, cosmetic or pharmaceutical active components, etc., may be used as examples of the auxiliaries and additives which may also be used in the microemulsions according to the invention. The type and quantity of components to be used is determined by the application envisaged for the emulsion and may readily be determined by the expert.

Commercial Applications

A preferred application for the microemulsion according to the invention is in the production of deodorizing cosmetic compositions, more particularly antiperspirant/deodorant sprays, antiperspirant/deodorant lotions, antiperspirant/deodorant roll-ons, antiperspirant/deodorant gels, antiperspirant/deodorant creams or antiperspirant/deodorant sticks.

Deodorants and Germ Inhibitors

Accordingly, microemulsions for this application preferably contain at least one deodorizing compound. This compound is selected in particular from germ-inhibiting compounds, enzyme inhibitors, more particularly trialkyl citrates, and astringent antiperspirants.

Basically, suitable germ inhibitors are any substances which act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), antibacterial perfumes, thymol, thyme oil, eugenol, clove oil, menthol, mint oil, farnesol, phenoxyethanol, glycerol monocaprate, glycerol monocaprylate, glycerol monolaurate (GML), diglycerol monocaprate (DMC), salicylic acid-N-alkylamides such as, for example, salicylic acid-n-octyl amide or salicylic acid-n-decyl amide.

Suitable enzyme inhibitors are, for example, esterase inhibitors. Esterase inhibitors are preferably trialkyl citrates, such as trimethyl citrate, tripropyl citrate, triisopropyl citrate, tributyl citrate and, in particular, triethyl citrate (Hydagen® CAT, Henkel KGaA, Dusseldorf, Del.). Esterase inhibitors inhibit enzyme activity and thus reduce odor formation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of ladanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic perfumes. Natural perfumes include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxy-citronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romilat, irotyl and floramat.

Antiperspirants are cosmetic preparations which reduce perspiration and thus reduce underarm wetness and contribute towards reducing body odor by influencing the activity of the eccrine sweat glands. The compounds preferably used in accordance with the invention are astringent antiperspirants, such as salts of aluminum, zirconium or zinc. Aluminum-zirconium salts in particular are distinguished by good antihydrotic activity. They are preferably complex compounds of amino acids, for example glycine, with chlorohydrate salts of the metals aluminum and zirconium in an Al:Zr ratio of 3 to 10:1 (ratio of metals to chloride 0.9 to 2.1:1). Aluminum-zirconium trichlorohydrate, aluminum-zirconium tetrachlorohydrate, aluminum-zirconium pentachlorohydrate, aluminum-zirconium octachlorohydrate and complex compounds thereof are preferably used.

Hitherto, antiperspirants have preferably been formulated as water-free suspensions or as water-containing solutions or emulsions. However, given a suitably high viscosity, as can also be achieved in the microemulsions according to the invention, the microemulsions according to the invention may also be directly used for processing into gels or sticks.

Thickeners

In another embodiment, at least one thickener, more particularly an associative thickener with a polar, hydrophilic middle part and nonpolar, hydrophobic end regions, is added to the microemulsion. This enables high viscosities to be established.

Among the usual thickeners, cellulose-based types are preferred. Thickeners with a polar middle part and nonpolar terminal regions are particularly advantageous. The middle part consists, for example, of a relatively long chain of ethylene oxide units (normally at least 80 units) which are esterified with fatty acids at the ends. Such compounds are normally referred to as “associative thickeners” because they are able to produce interactions between oil droplets with their nonpolar ends. Examples of such thickeners are Eumulgin® EO 33 (PEG-150 Distearate) and Eumulgin® EO 35 (PEG-140 Glyceryl Tristearate) from Cognis Deutschland GmbH & Co. KG, PEG-175 Distearate (AEC PEG-175 distearate) from A. & E. Connock, PEG-190 Distearate (Emulmin® 862) from Sanyo Chemicals, PEG-250 Distearate (Emanon® 3299R) from Kao, PEG-150 Dibehenate (Ethox® P-6000 DB) from Ethox Chemicals, PEG-90 Diisostearate (Hydramol® PGDS) from Scher Chemicals, PEG-175 Diisostearate (Ethox® HVB) from Ethox Chemicals, PEG-150 Dioleate (AEC® PEG-150 Dioleate) from A. & E. Connock, PEG-150 Pentaerythrityl Tetrastearate (Crothix®) from Croda, PEG-120 Methyl Glucose Trioleate (Glucamate® LT) from Amerchol Corporation, PEG-120 Methyl Glucose Dioleate (Glucamate® DOE-120) from Degussa Care Specialties and Antil® (Antil® 120, Antil® 127) from Degussa Care Specialties.

Even where these thickeners and deodorizing/antiperspirant components are incorporated in the microemulsions according to the invention, transparent or translucent emulsions which produce a barely noticeable feeling of tackiness, if any at all, on the skin are obtained in contrast to the prior art, where non-transparent and tacky emulsions are often formed in such cases. Among the thickeners, PEG-150 Dibehenate and PEG-90 Diisostearate are particularly preferred. By comparison with the other associative thickeners, PEG-150 Dibehenate provides surprisingly high gel viscosities. Formulations containing this associative thickener are bright and clear, even with a high content of the antiperspirant salts, have a very pleasant feeling on the skin.

The invention is illustrated by the following Formulation Examples.

### EXAMPLES

Microemulsions were prepared to the formulations shown in Table 1. To this end, the oil-soluble constituents were heated to ca. 85° C., melted and homogeneously stirred. The water phase containing the water-soluble constituents and heated to the same temperature was partly incorporated in the oil phase by stirring and the formulation was cold-stirred. The quantities shown represent % by weight of the commercially available substances in the composition as a whole.

TABLE 1

| INGREDIENTS | Ex. 1 | Ex. 2 | Ex. 3 | Ex. 4 | Ex. 5 | Ex. 6 | Ex. 7 | Ex. 8 |
|---|---|---|---|---|---|---|---|---|
| INCI (trade name) | Wt. % | Wt. % | Wt. % | Wt. % | Wt. % | Wt. % | Wt. % | Wt. % |
| Ceteareth-12 (Eumulgin ® B1) | 0.9 | 0.8 | 0.8 | 0.8 | 1.0 | 0.9 | 0.9 | 0.4 |
| Ceteareth-20 (Eumulgin ® B2) | 7.5 | 6.8 | 1.6 | 6.9 | 7.7 | 7.7 | 7.7 | 3.9 |
| Beheneth-25 (Eumulgin ® BA 25) | — | — | 5.2 | — | — | — | — | — |
| Eumulgin ® HRE 40 | 11.3 | 10.3 | — | — | — | — | — | — |
| Eumulgin ® L | — | — | — | 3.4 | 4.3 | 4.3 | 4.3 | 1.7 |
| PPG-5-Laureth-5 (Aethoxal ® B) | — | — | 10.3 | — | — | — | — | — |
| Glyceryl Stearate (Cutina ® GMS) | 4.5 | 4.2 | 4.2 | 3.9 | 4.9 | 4.3 | 4.3 | 2.0 |
| Cetearyl Alcohol (Lanette ® O) | 0.9 | 0.8 | 0.8 | 0.8 | 1.0 | 0.9 | 0.9 | 0.4 |
| Cetyl Palmitate (Cutina ® CP) | 0.9 | 0.8 | 0.8 | 0.8 | 1.0 | 0.9 | 0.9 | 0.4 |
| Cyclomethicone (DC ® 245) | 9.5 | 8.7 | 8.7 | 7.0 | 9.0 | 8.7 | 8.7 | 3.5 |
| Dicaprylyl Carbonate (Cetiol ® CC) | 7.0 | — | — | — | — | — | — | — |
| Dicaprylyl Ether (Cetiol ® OE) | — | 6.5 | 6.5 | 5.1 | 6.5 | 6.5 | 6.5 | 2.6 |
| Diethylhexylcyclohexan (Cetiol ® S) | 9.5 | 8.7 | 8.7 | 7.0 | 9.0 | 8.7 | 8.7 | 3.5 |
| Paraffinum Liquidum | — | — | — | — | — | 1.0 | — | — |
| Stearyl Dimethicone (DC ® 2503) | — | — | — | — | — | 1.0 | — | — |
| Ethanol, cosmet. | — | — | 5.0 | — | — | — | — | — |
| Panthenol | — | — | — | — | — | 0.5 | — | — |
| Glycerol | — | — | — | — | — | 1.0 | — | — |
| Triclosan (Irgasan ® DP 300) | — | 0.1 | — | — | — | — | — | — |
| Triethyl Citrate (Hydagen ® CAT) | — | 0.5 | — | — | — | — | — | — |
| Aluminum Chlorohydrate (Locron ® L) | — | — | 40.0 | — | — | — | — | — |
| Aluminum Zirconium Tetrachloro- | 40.0 | 30.0 | — | 40.0 | 40.0 | — | 40.0 | 40.0 |
| hydrex Gly (Rezal ® 36 GC) | | | | | | | | |
| Aluminum Zirconium Pentachloro- | — | — | — | — | — | 40.0 | — | — |
| hydrate (Rezal ® 67) | | | | | | | | |
| Hydroxyethylcellulose (Natrosol ® | — | — | — | 1.0 | — | — | — | — |
| 250 HHR) | | | | | | | | |
| Aqua | To 100 | To 100 | To 100 | To 100 | To 100 | To 100 | To 100 | To 100 |
| Used as | Cream | Cream | Cream | Solid cream | Soft | Lotion | Lotion | Spray |
| | | | | | cream | | | |
| Viscosity (mPa · s) | 800.000 | 300.000 | 300.000 | >1.000.000 | 80.000 | 10.000 | 5.000 | <400 |

| INGREDIENTS | Ex. 9 | Ex. 10 | Ex. 11 | Ex. 12 | Ex. 13 | Ex. 14 |
|---|---|---|---|---|---|---|
| INCI (trade name) | Wt. % | Wt. % | Wt. % | Wt. % | Wt. % | Wt. % |
| Ceteareth-12 (Eumulgin ® B1) | 0.3 | 0.3 | 0.4 | 0.4 | 0.4 | 0.4 |
| Ceteareth-20 (Eumulgin ® B2) | 2.2 | 0.6 | 0.8 | 0.8 | 0.8 | 0.8 |
| Beheneth-25 (Eumulgin ® BA 25) | — | 2.6 | 3.2 | 3.2 | 3.2 | 3.2 |
| Ceteareth-30 (Eumulgin ® B3) | — | — | — | 0.2 | — | — |
| Eumulgin ® L | 1.3 | 2.3 | 2.9 | 4.4 | 3.3 | 3.3 |
| Glyceryl Stearate (Cutina ® GMS) | 1.7 | 1.7 | 2.1 | 2.1 | 2.1 | 2.1 |
| Cetearyl Alcohol (Lanette ® O) | 0.3 | 0.3 | 0.4 | 0.4 | 0.4 | 0.4 |
| Cetyl Palmitate (Cutina ® CP) | 0.3 | 0.3 | 0.4 | 0.4 | 0.4 | 0.4 |
| Cyclomethicone (DC ® 245) | 2.6 | 2.6 | 1.8 | 1.8 | 1.8 | 1.8 |
| Dicaprylyl Carbonate (Cetiol ® CC) | — | 1.9 | — | — | — | — |
| Dicaprylyl Ether (Cetiol ® OE) | 1.9 | — | 1.4 | 1.4 | 1.4 | 1.4 |
| Diethylhexylcyclohexan (Cetiol ® S) | 2.6 | 1.6 | 1.9 | 1.9 | 1.9 | 1.9 |
| Paraffinum Liquidum | — | 1.0 | — | — | — | — |
| PEG-150 Distearate | — | — | — | 0.5 | 1.5 | — |
| (Eumulgin ® EO 33) | | | | | | |
| PEG-140 Glyceryl Tristearate | — | — | — | — | — | 1.5 |
| (Eumulgin ® EO 35) | | | | | | |
| Aluminum Chlorohydrate | 20.0 | — | 20.0 | — | — | — |
| (Locron ® L) | | | | | | |
| Aluminum ZirconiumTetrachloro- | — | 20.0 | — | 20.0 | 20.0 | 20.0 |
| hydrex Gly (Rezal ® 36 GC) | | | | | | |
| Aqua | to 100 | to 100 | to 100 | to 100 | to 100 | to 100 |
| Used as | Spray | Spray | Spray | Lotion | Cream | Cream |
| Viscosity (mPa · s) | <400 | <400 | <400 | 1000 | 300.000 | 300.000 |
| INGREDIENTS | Ex. 15 | Ex. 16 | Ex. 17 | Ex. 18 | Ex. 19 | Ex. 20 |
| INCI (trade name) | Wt. % | Wt. % | Wt. % | Wt. % | Wt. % | Wt. % |
| Ceteareth-12 (Eumulgin ® B1) | 0.4 | 0.4 | 0.4 | 0.4 | 0.4 | 0.4 |
| Ceteareth-20 (Eumulgin ® B2) | 0.8 | 0.8 | 0.8 | 0.7 | 0.8 | 0.8 |
| Beheneth-25 (Eumulgin ® BA 25) | 3.2 | 3.2 | 3.2 | 3.7 | 3.2 | 3.2 |
| Ceteareth-30 (Eumulgin ® B3) | 0.4 | 0.4 | 0.4 | 0.4 | 0.4 | 0.4 |
| Eumulgin ® L | 4.4 | 4.4 | 4.4 | 4.4 | 4.4 | 4.4 |
| Glyceryl Stearate (Cutina ® GMS) | 2.1 | 2.1 | 2.1 | 1.8 | 2.1 | 2.1 |
| Cetearyl Alcohol (Lanette ® O) | 0.4 | 0.4 | 0.4 | 0.3 | 0.4 | 0.4 |
| Cetyl Palmitate (Cutina ® CP) | 0.4 | 0.4 | 0.4 | 0.4 | 0.4 | 0.4 |
| Cydomethicone (DC ® 245) | 1.8 | 1.8 | 1.8 | 1.8 | 1.8 | 1.8 |
| Dicaprylyl Carbonate (Cetiol ® CC) | — | — | — | — | — | — |
| Dicaprylyl Ether (Cetiol ® OE) | 1.4 | 1.4 | 1.4 | 1.4 | 1.4 | 1.4 |
| Diethylhexylcyclohexan (Cetiol ® S) | 1.9 | 1.9 | 1.9 | 1.9 | 1.9 | 1.9 |
| PEG-175 Distearate | 0.5 | — | — | — | — | — |
| (AEC PEG-175 Distearate) | | | | | | |
| PEG-190 Distearate | — | 0.4 | — | — | — | — |
| (Emulmin ® 862) | | | | | | |
| PEG-250 Distearate | — | — | 0.4 | — | — | — |
| (Emanon ® 3299R) | | | | | | |
| PEG-150 Dibehenate | — | — | — | 0.5 | — | — |
| (Ethox ® P-6000 DB) | | | | | | |
| PEG-90 Diisostearate | — | — | — | — | 0.7 | — |
| (Hydramol ® PGDS) | | | | | | |
| PEG-175 Diisostearate | — | — | — | — | — | 1.0 |
| (Ethox ® HVB) | | | | | | |
| Aluminum ZirconiumTetrachloro- | — | — | — | 20.0 | 20.0 | 20.0 |
| hydrex Gly (Rezal ® 36 GC) | | | | | | |
| Aluminum Chlorohydrate | 20.0 | 20.0 | 20.0 | — | — | — |
| (Locron ® L) | | | | | | |
| Aqua | to 100 | to 100 | to 100 | to 100 | to 100 | to 100 |
| Used as | Lotion | Lotion | Lotion | Cream | Lotion | Soft |
| | | | | | | cream |
| Viscosity (mPa · s) | <2000 | <2000 | <2000 | 90.000 | <1000 | 16.000 |

| INGREDIENTS | Ex. 21 | Ex. 22 | Ex. 23 | Ex. 24 |
|---|---|---|---|---|
| INCI (trade name) | Wt. % | Wt % | Wt. % | Wt. % |
| Ceteareth-12 (Eumulgin ® B1) | 0.4 | 0.4 | 0.4 | 0.4 |
| Ceteareth-20 (Eumulgin ® B2) | 0.8 | 0.8 | 0.8 | 0.8 |
| Beheneth-25 (Eumulgin ® BA 25) | 3.2 | 3.2 | 3.2 | 3.2 |
| Ceteareth-30 (Eumulgin ® B3) | 0.4 | 0.4 | 0.4 | 0.4 |
| Eumulgin ® L | 4.4 | 4.4 | 4.4 | 4.4 |
| Glyceryl Stearate (Cutina ® GMS) | 2.1 | 2.1 | 2.1 | 2.1 |
| Cetearyl Alcohol (Lanette ® O) | 0.4 | 0.4 | 0.4 | 0.4 |
| Cetyl Palmitate (Cutina ® CP) | 0.4 | 0.4 | 0.4 | 0.4 |
| Cyclomethicone (DC ® 245) | 1.8 | 1.8 | 1.8 | 1.8 |
| Dicaprylyl Carbonate (Cetiol ® CC) | — | — | — | — |
| Dicaprylyl Ether (Cetiol ® OE) | 1.4 | 1.4 | 1.4 | 1.4 |
| Diethylhexylcyclohexan (Cetiol ® S) | 1.9 | 1.9 | 1.9 | 1.9 |
| PEG-150 Dioleate | 0.5 | — | — | — |
| (AEC ® PEG-150 Dioleate) | | | | |
| PEG-150 Pentaerythrityl | — | 0.7 | — | — |
| Tetrastearate | | | | |
| (Crothix ®) | | | | |
| PEG-120 Methyl Glucose Dioleate | — | — | 2.0 | — |
| (Glucamate ® DOE-120) | | | | |
| PEG-120 Methyl Glucose Trioleate | — | — | — | 4.0 |
| (Glucamate ® LT) | | | | |
| Aluminum ZirconiumTetrachloro- | — | — | 20.0 | 20.0 |
| hydrex Gly (Rezal ® 36 GC) | | | | |
| Aluminum Chlorohydrate | 20.0 | 20.0 | — | — |
| (Locron ® L) | | | | |
| Aqua | to 100 | to 100 | to 100 | to 100 |
| Used as | Lotion | Soft cream | Lotion | Lotion |
| Viscosity (mPa · s) | <2000 | 26.000 | <1000 | <1000 |

The viscosity of the formulations was determined by measurement with a Brookfield viscosimeter at 23° C. Where the viscosities are shown in italics, a Brookfield RVF viscosimeter with a Helipath TF spindle, 4 r.p.m., was used; where the viscosities are shown in bold type, they were measured with a Brookfield RVT viscosimeter with a Helipath TC spindle at 5 r.p.m. The underlined viscosities were measured with a Brookfield RVF viscosimeter with spindle 5 at 10 r.p.m.

The formulations had a transmission of 97 to 100% (wave length: 700 nm; 1 cm layer thickness of the cell; 23° C.; Spectronic 20, manufacturer: Milton Roy Company) and mean droplet sizes below 100 nm.

The microemulsion of Example 12 was used to produce a roll-on deodorant stick and, in a sensory test, was compared by trained testers with a roll-on deodorant stick that had been produced in accordance with the prior art. To this end, a deodorizing preparation substantially corresponding to Example 1 of WO-A-96/28131 was prepared. This preparation was adjusted to a viscosity corresponding to Example 12 simply by adding 0.5% by weight of the thickener present in Example 12, Eumulgin® EO 33. Accordingly, the composition of the Comparison Example was as follows:

Comparison Example

| Ingredients | % by weight |
|---|---|
| Glyceryl isostearate | 18 |
| PEG-15-cetyl stearyl alcohol | 5.1 |
| Octyl isostearate | 3.3 |
| Cyclomethicone | 6.6 |
| Sorbitol | 2.9 |
| Glycerol monocaprate | 0.1 |
| Aluminum chlorohydrate | 3.9 |
| PEG-150 Distearate (Eumulgin ® EO 33) | 0.5 |
| Water | 75.8 |

The lotions were applied to the forearm and the following criteria were evaluated on a scale of −2 to +2: spreading (−2: difficult to +2: easy), adsorption (−2: difficult to +2: easy), tackiness (−2: high to +2: low), oiliness (−2: high to +2: low), waxiness (−2: high to +2: low), smoothness (−2: low to +2: high), softness (−2: low to +2: high) and dryness (−2: low to +2: high). The results are set out in the following Table as mean values of the individual evaluations.

TABLE 2

| | Spreading | Adsorption | Tackiness | Oiliness | Waxiness | Smoothness | Softness | Dryness |
|---|---|---|---|---|---|---|---|---|
| Roll-on lotion of Ex. 12 | +1 | +1 | +1 | +1 | +1 | +1 | +1 | +1 |
| Roll-on lotion of Comp. Ex. | 0 | 0 | 0 | 0 | 0 | 0 | 0 | 0 |

### APPENDIX

1) AEC® PEG-150 Dioleate

INCI: PEG-150 Dioleate

Manufacturer: A & E Connock

2) AEC® PEG-175 Distearat

INCI: PEG-175 Distearat

Manufacturer: A & E Connock

3) Aethoxal® B

INCI: PPG-5-Laureth-5

Manufacturer: Cognis Deutschland GmbH & Co. KG

4) Cetiol® OE

INCI: Dicaprylyl Ether

Manufacturer: Cognis Deutschland GmbH & Co. KG

5) Cetiol® CC

INCI: Dicaprylyl Carbonate

Manufacturer: Cognis Deutschland GmbH & Co. KG

6) Cetiol® S

INCI: Diethylhexylcyclohexan

Manufacturer: Cognis Deutschland GmbH & Co. KG

7) Crothix®

INCI: PEG-150 Pentaerythrityl Tetrastearat

Manufacturer: Croda

8) Cutina® CP

INCI: Cetyl Palmitate

Manufacturer: Cognis Deutschland GmbH & Co. KG

9) Cutina® GMS

INCI: Glyceryl Stearate

Manufacturer: Cognis Deutschland GmbH & Co. KG

10) Dow Corning® 245

INCI: Cyclomethicone

Manufacturer: Dow Corning

11) Dow Corning® 2503

INCI: Stearyl Dimethicone

Manufacturer: Dow Corning

12) Emanon® 3299R

INCI: PEG-250 Distearat

Manufacturer: Kao

13) Emulmin®862

INCI: PEG-190 Distearat

Manufacturer: Sanyo Chemicals

14) Ethox® HVB

INCI: PEG-175 Diisostearat

Manufacturer: Ethox Chemicals

15) Ethox® P-6000 DB

INCI: PEG-150 Dibehenat

Manufacturer: Ethox Chemicals

16) Eumulgin® B1

INCI: Ceteareth-12

Manufacturer: Cognis Deutschland GmbH & Co. KG

17) Eumulgin®B2

INCI: Ceteareth-20

Manufacturer: Cognis Deutschland GmbH & Co. KG

18) Eumulgin® B3

INCI: CETEARETH-30

Manufacturer: Cognis Deutschland GmbH & Co. KG

19) Eumulgin® BA 25

INCI: BEHENETH-25

Manufacturer: Cognis Deutschland GmbH & Co. KG

20) Eumulgin® EO 33

INCI: PEG-150 DISTEARAT

Manufacturer: Cognis Deutschland GmbH & Co. KG

21) Eumulgin® EO 35

INCI: PEG-140 GLYCERYL TRISTEARAT

Manufacturer: Cognis Deutschland GmbH & Co. KG

22) Eumulgin® HRE 40

INCI: PEG-40 Hydrogenated castor oil

Manufacturer: Cognis Deutschland GmbH & Co. KG

23) Eumulgin® L

INCI: PPG-1-PEG-9 Lauryl Glycol Ether

Manufacturer: Cognis Deutschland GmbH & Co. KG

24) Glucamate® DOE-120

INCI: PEG-120 Methyl Glucose Dioleate

Manufacturer: Degussa Care Specialties

25) Glucamate® LT

INCI: PEG-120 Methyl Glucose Trioleate

Manufacturer: Amerchol Corporation

26) Hydagen® CAT

INCI: Triethyl Citrate

Manufacturer: Cognis Deutschland GmbH & Co. KG

27) Hydramol® PGDS

INCI: PEG-90 Diisostearat

Manufacturer: Scher Chemicals

28) Irgasan® DP300

INCI: Triclosan

Manufacturer: Ciba

29) Lanette® O

INCI: Cetearyl Alcohol

Manufacturer: Cognis Deutschland GmbH & Co. KG

30) Locron® L

INCI: Aluminum Chlorohydrate

Manufacturer: Clariant

31) Natrosol® 250 HHR

INCI: Hydroxyethylcellulose

Manufacturer: Hercules

32) Rezal® 36GC

INCI: Aluminum Zirconium Tetrachlorohydrex Gly

Manufacturer: Reheis (Interorgana)

33) Rezal® 67

INCI: Aluminum Zirconium Pentachlorohydrate

Manufacturer: Reheis (Interorgana)

1-15. (canceled)

16. An emulsifier composition comprising hydrophilic and lipophilic emulsifiers, wherein the hydrophilic emulsifier is selected from at least one of the compounds of group (A) which consists of
(A.1) products of the addition of 8 to 30 moles of ethylene oxide onto C₁₆₋₂₂ fatty alcohols,
(A.2) products of the addition of 5 to 60 moles of ethylene oxide onto C₁₂₋₁₈ fatty acid triglycerides,
(A.3) products of the addition of 10 to 30 moles of ethylene oxide onto sorbitan mono-C₁₂₋₁₈-fatty acid esters,
(A.4) products of the addition of 10 to 30 moles of ethylene oxide onto C₈₋₁₈ fatty acid partial glycerides and
(A.5) products of the addition of 1 to 10 moles of ethylene oxide and 1 to 5 moles of propylene oxide onto C₁₀₋₁₈ fatty alcohols,
and wherein the lipophilic emulsifier is selected from at least one of the compounds of group (B) which consists of
(B.1) mono- and diesters of C₁₆₋₁₈ fatty acids with glycerol and
(B.2) C₁₆₋₂₂ fatty alcohols,
with the proviso that, based on the total quantity of hydrophilic emulsifier, at least 38% by weight of the compounds selected from group (A) are emulsifiers formed by addition of at least 20 moles of ethylene oxide and that the hydrophilic emulsifier is present in a quantity of 65 to 80% by weight, based on the total quantity of hydrophilic and lipophilic emulsifiers.

17. The emulsifier composition according to claim 16, wherein the hydrophilic emulsifier is selected from at least one of the compounds of group (A) which consists of
(A.1) products of the addition of 10 to 25 moles of ethylene oxide onto C₁₆₋₂₂ fatty alcohols,
(A.2) reaction products of 30 to 60 moles of ethylene oxide with castor oil, hydrogenated castor oil or almond oil,
(A.3) products of the addition of 15 to 25 moles of ethylene oxide onto sorbitan mono-C₁₆₋₂₂ fatty acid esters,
(A.4) products of the addition of 15 to 25 moles of ethylene oxide onto C₈₋₁₂ fatty acid partial glycerides, and
(A.5) products of the addition of 1 to 10 moles of ethylene oxide and 1 to 5 moles of propylene oxide onto C₁₀₋₁₄ fatty alcohols.

18. The emulsifier composition according to claim 17, wherein components A.3 and A.5, if present, are selected from
(A.3) products of the addition of 15 to 25 moles of ethylene oxide onto sorbitan mono-C₁₈-fatty acid esters, and
(A.5) products of the addition of 1 to 10 moles of ethylene oxide and 1 to 5 moles ofpropylene oxide onto C₁₂ fatty alcohols.

19. The emulsifier composition according to claim 16, wherein the hydrophilic emulsifier is selected from at least one of the compounds of the group of A.1, A.2 and A.5 components consisting of
(A.1) products of the addition of 10 to 30 moles of ethylene oxide onto cetyl alcohol, stearyl alcohol and mixtures thereof, and behenyl alcohol,
(A.2) products of the reaction of 40 to 60 moles of ethylene oxide with castor oil or hydrogenated castor oil and
(A.5) products of the addition of 5 to 10 moles of ethylene oxide and 1 to 5 moles of propylene oxide onto lauryl alcohol.

20. The emulsifier composition according to claim 16, wherein the lipophilic emulsifier is selected from at least one of the compounds of the group B.1 and B.2 components consisting of
(B.1) partial glycerides of stearic acid and
(B.2) cetyl alcohol, stearyl alcohol and mixtures thereof, and behenyl alcohol.

21. The emulsifier composition according to claim 16, wherein, based on the total quantity of hydrophilic emulsifier, 47 to 96% by weight of the compounds selected from group (A) are hydrophilic emulsifiers formed by addition of at least 20 moles of ethylene oxide.

22. The emulsifier composition according to claim 16, wherein the hydrophilic emulsifier is selected from at least one of the compounds of the group A.1, A.2 and A.5 components consisting of
(A.1) products of the addition of 10 to 30 moles of ethylene oxide onto cetyl alcohol, stearyl alcohol, behenyl alcohol and mixtures thereof,
(A.2) products of the reaction of 40 to 60 moles of ethylene oxide with castor oil or hydrogenated castor oil and
(A.3) products of the addition of 5 to 10 moles of ethylene oxide and 1 to 5 moles of propylene oxide onto lauryl alcohol; and
the lipophilic emulsifier is selected from at least one of the compounds of the group of B.1 and B.2 components consisting of
(B.1) partial glycerides of stearic acid and
(B.2) cetyl alcohol, stearyl alcohol and mixtures thereof, and behenyl alcohol.

23. The emulsifier composition according to claim 17, wherein the hydrophilic emulsifier is present in a quantity of 70 to 80% by weight, based on the total quantity of hydrophilic and lipophilic emulsifiers.

24. The emulsifier composition as according to claim 22, wherein the hydrophilic emulsifier is present in a quantity of 70 to 80% by weight, based on the total quantity of hydrophilic and lipophilic emulsifiers.

25. A microemulsion comprising an emulsifying agent comprising hydrophilic and lipophilic emulsifiers, wherein the hydrophilic emulsifier is selected from at least one of the compounds of group (A) which consists of
(A.1) products of the addition of 8 to 30 moles of ethylene oxide onto C₁₆₋₂₂ fatty alcohols,
(A.2) products of the addition of 5 to 60 moles of ethylene oxide onto C₁₂₋₁₈ fatty acid triglycerides,
(A.3) products of the addition of 10 to 30 moles of ethylene oxide onto sorbitan mono-C₁₂₋₁₈-fatty acid esters,
(A.4) products of the addition of 10 to 30 moles of ethylene oxide onto C₈₋₁₈ fatty acid partial glycerides and
(A.5) products of the addition of 1 to 10 moles of ethylene oxide and 1 to 5 moles of propylene oxide onto C₁₀₋₁₈ fatty alcohols,
and wherein the lipophilic emulsifier is selected from at least one of the compounds of group (B) which consists of
(B.1) mono- and diesters of C₁₆₋₁₈ fatty acids with glycerol and
(B.2) C₁₆₋₂₂ fatty alcohols,
with the proviso that, based on the total quantity of hydrophilic emulsifier, at least 38% by weight of the compounds selected from group (A) are emulsifiers formed by addition of at least 20 moles of ethylene oxide and that the hydrophilic emulsifier is present in a quantity of 65 to 80% by weight, based on the total quantity of hydrophilic and lipophilic emulsifiers.

26. The microemulsion according to claim 25, wherein the hydrophilic emulsifier is selected from at least one of the compounds of group (A) which consists of
(A.1) products of the addition of 10 to 25 moles of ethylene oxide onto C₁₆₋₂₂ fatty alcohols,
(A.2) reaction products of 30 to 60 moles of ethylene oxide with castor oil, hydrogenated castor oil or almond oil,
(A.3) products of the addition of 15 to 25 moles of ethylene oxide onto sorbitan mono-C₁₆₋₁₈-fatty acid esters,
(A.4) products of the addition of 15 to 25 moles of ethylene oxide onto C₈₋₁₂ fatty acid partial glycerides, and
(A.5) products of the addition of 1 to 10 moles of ethylene oxide and 1 to 5 moles of propylene oxide onto C₁₀₋₁₄ fatty alcohols.

27. The microemulsion according to claim 26, wherein components A.3 and A.5, if present are selected from
(A.3) products of the addition of 15 to 25 moles of ethylene oxide onto sorbitan mono-C₁₈-fatty acid esters, and
(A.5) products of the addition of 1 to 10 moles of ethylene oxide and 1 to 5 moles ofpropylene oxide onto C₁₂ fatty alcohols.

28. The microemulsion according to claim 25, wherein the hydrophilic emulsifier is selected from at least one of the compounds of the group of A.1, A.2 and A.5 components consisting of
(A.1) products of the addition of 10 to 30 moles of ethylene oxide onto cetyl alcohol, stearyl alcohol and mixtures thereof, and behenyl alcohol,
(A.2) products of the reaction of 40 to 60 moles of ethylene oxide with castor oil or hydrogenated castor oil and
(A.5) products of the addition of 5 to 10 moles of ethylene oxide and 1 to 5 moles of propylene oxide onto lauryl alcohol.

29. The microemulsion according to claim 25, wherein the lipophilic emulsifier is selected from at least one of the compounds of the group B.1 and B.2 components consisting of
(B.1) partial glycerides of stearic acid and
(B.2) cetyl alcohol, stearyl alcohol and mixtures thereof, and behenyl alcohol.

30. The microemulsion composition according to claim 25, wherein the hydrophilic emulsifier is present in a quantity of 70 to 80% by weight, based on the total quantity of hydrophilic and lipophilic emulsifiers.

31. The microemulsion according to claim 25, wherein the emulsifier composition is present in a quantity of 5 to 30% by weight, based on the total quantity of the microemulsion.

32. The transparent or translucent microemulsion according to claim 25, wherein the emulsifier composition is present in a quantity of 10 to 25% by weight, based on the total quantity of the microemulsion.

33. The microemulsion according to claim 25, which is transparent or translucent.

34. The microemulsion according to claim 25, comprising an aqueous phase and an oil phase, the latter having components selected from one or more of oils, fats and waxes in a quantity of 30 to 60% by weight, based on the total quantity of oil components, fats and waxes and the emulsifier composition.

35. The microemulsion according to claim 25, further comprising components selected from one or more oils, fats and waxes in a quantity of 49 to 58% by weight, based on the total quantity of oil components, fats and waxes and the emulsifier composition.

36. The microemulsion according to 34, comprising an oil phase and an aqueous phase in a quantity ratio of 10 to 55% by weight: 90 to 45% by weight.

37. The microemulsion according to 34, comprising an oil phase and an aqueous phase in a quantity ratio of 15 to 50% by weight: 85 to 50% by weight.

38. The microemulsion according to claim 34, comprising, as the oil component, at least one compound selected from the group consisting of
(a) esters of a C₁₆₋₂₂ fatty acid with a C₈₋₁₈ alcohol,
(b) dialkyl carbonates with C₆₋₂₂ alkyl groups,
(c) silicone oils,
(d) aliphatic or naphthenic hydrocarbons, and
(e) symmetrical or nonsymmetrical dialkyl ethers with C₆₋₂₂ alkyl groups.

39. The microemulsion according to claim 34, comprising, as the oil component, at least one compound selected from the group consisting of
(a) esters of a linear C₁₆₋₂₂ fatty acid with a linear C₈₋₁₈ alcohol,
(b) dialkyl carbonates with linear C₆₋₁₂ alkyl groups,
(c) silicone oils,
(d) dialkyl cyclohexanes, and
(e) symmetrical or nonsymmetrical dialkyl ethers with linear C₆₋₁₂ alkyl groups.

40. The microemulsion according to claim 25, further comprising at least one deodorizing compound selected from
(a) germ-inhibiting compounds,
(b) enzyme inhibitors,
(c) astringent antiperspirants.

41. The microemulsion according to claim 39, wherein components (b) and (c) are selected from
(b) trialkyl citrates, and
(c) aluminum-zirconium trichlorohydrate, aluminum-zirconium tetrachlorohydrate, aluminum-zirconium pentachlorohydrate, aluminum-zirconium octachloro hydrate and complex compounds thereof.

42. The microemulsion according to claim 25, further comprising at least one associative thickener with a polar, hydrophilic middle region and nonpolar, hydrophobic end regions.

43. A deodorizing cosmetic composition, comprising
(1) a translucent or transparent microemulsion containing an emulsifying agent comprising hydrophilic and lipophilic emulsifiers, wherein the hydrophilic emulsifier is selected from at least one of the compounds of group (A) which consists of
(A.1) products of the addition of 8 to 30 moles of ethylene oxide onto C₁₆₋₂₂ fatty alcohols,
(A.2) products of the addition of 5 to 60 moles of ethylene oxide onto C₁₂₋₁₈ fatty acid triglycerides,
(A.3) products of the addition of 10 to 30 moles of ethylene oxide onto sorbitan mono-C₁₂₋₁₈-fatty acid esters,
(A.4) products of the addition of 10 to 30 moles of ethylene oxide onto C₈₋₁₈ fatty acid partial glycerides and
(A.5) products of the addition of 1 to 10 moles of ethylene oxide and 1 to 5 moles of propylene oxide onto C₁₀₋₁₈ fatty alcohols,
and wherein the lipophilic emulsifier is selected from at least one of the compounds of group (B) which consists of
(B.1) mono- and diesters of C₁₆₋₁₈ fatty acids with glycerol and
(B.2) C₁₆₋₂₂ fatty alcohols,
with the proviso that, based on the total quantity of hydrophilic emulsifier, at least 38% by weight of the compounds selected from group (A) are emulsifiers formed by addition of at least 20 moles of ethylene oxide and that the hydrophilic emulsifier is present in a quantity of 65 to 80% by weight, based on the total quantity of hydrophilic and lipophilic emulsifiers; and
(2) at least one deodorizing compound selected from
(a) germ-inhibiting compounds,
(b) enzyme inhibitors, and
(c) astringent antiperspirants.

44. The deodorizing composition according to claim 43, wherein the hydrophilic emulsifier is selected from at least one of the compounds of the group A.1, A.2 and A.5 components consisting of
(A.1) products of the addition of 10 to 30 moles of ethylene oxide onto cetyl alcohol, stearyl alcohol, behenyl alcohol and mixtures thereof,
(A.2) products of the reaction of 40 to 60 moles of ethylene oxide with castor oil or hydrogenated castor oil and
(A.3) products of the addition of 5 to 10 moles of ethylene oxide and 1 to 5 moles of propylene oxide onto lauryl alcohol; and
the lipophilic emulsifier is selected from at least one of the compounds of the group of B.1 and B.2 components consisting of
(B.1) partial glycerides of stearic acid and
(B.2) cetyl alcohol, stearyl alcohol and mixtures thereof, and behenyl alcohol.

45. The deodorizing composition according to claim 43, wherein deodorizing compounds (b) and (c) are selected from
(b) trialkyl citrates, and
(c) aluminum-zirconium trichlorohydrate, aluminum-zirconium tetrachlorohydrate, aluminum-zirconium pentachlorohydrate, aluminum-zirconium octachloro hydrate and complex compounds thereof.

46. The deodorizing composition according to claim 43 in the form of a spray, a lotion, a roll-on, a cream or a stick.
